(19) United States
(12) Patent Application Publication
(10) Pub. No.: US 2020/0367753 A1
(43) Pub. Date: Nov. 26, 2020

(54) METHOD AND DEVICE FOR MONITORING THE FLUORESCENCE EMITTED AT THE SURFACE OF A BIOLOGICAL TISSUE

(71) Applicant: TRUMPF Schweiz AG, Gruesch (CH)

(72) Inventors: Anthony Daures, Clichy (FR); Philippe Rizo, La Tronche (FR)

(21) Appl. No.: 16/757,567

(22) PCT Filed: Oct. 24, 2018
(86) PCT No.: PCT/FR2018/052639

§ 371 Date: Apr. 20, 2020

(30) Foreign Application Priority Data
Oct. 26, 2017 (FR) 1706011

(51) Int. Cl. A61B 5/00; A61B 90/00

(52) U.S. Cl. CPC A61B 5/0071; A61B 5/0077; H04N 5/2256; A61B 90/39; A61B 2090/3941; A61B 5/742

(57) ABSTRACT

A method for monitoring diffusion over time of a fluorescent marker that has been injected into a biological tissue at an injection-time includes using an excitation light source, exciting the fluorescent marker and, during an interval that begins after the injection-time and ends at an end-time, using a camera to acquire fluorescence images of an area of the biological tissue. This includes executing first and second image-acquisition sequences starting at different start-times after the injection time. The method includes comparing first and second images that result from having processed the image-acquisition sequences.

### RELATED APPLICATIONS

This application is the national stage under § 371 of international application PCT/FR2018/052639, filed on Oct. 24, 2018, which claims the benefit of the priority date of French application FR1760111, Oct. 26, 2017, the contents of which are incorporated by reference.

### FIELD OF INVENTION

The invention relates to medical imaging and in particular, to fluorescence in biological tissue.

### BACKGROUND

Fluorescence imaging provides pre-operative information regarding perfusion in biological tissues, and in particular, human tissues.

In some cases, it is useful to identify and locate those areas in which a fluorescence signal first appears and to observe rates at which that signal changes over time. This can be useful for evaluating perfusion into tissue. Such evaluation is useful for identifying vascular problems.

### SUMMARY

The method described herein creates the possibility of directly viewing, on an image of the observed biological tissue, the result of a computational operation that includes pixel-by-pixel comparison of two images representative of the diffusion observed at successive times. This permits the viewer to see the evolution of fluorescence provided by the fluorescent marker.

The image resulting from this computational operation reveals local variation in a fluorescence signal at different measurement times for the entire observed area of the tissue, and not merely for a restricted area or for a few pixels. Thus, it becomes possible to easily observe diffusion of the marker through, for example, an entire foot. This makes it possible to follow the progress of perfusion by viewing how this variation changes over time and locally (by viewing the variation in the signal for each pixel but for all the pixels of the image). As a result, it becomes possible to view the progress of perfusion into an area and into the environment around that area. This makes it possible to determine if a signal is increasing around a certain area but not increasing within another area. Such an observation may indicate, for example, a blockage in the artery that vascularizes that area.

Images obtained according to methods described herein are useful in a variety of applications, including the treatment of chronic wounds. Such images are easily screened by technicians who can then call for prompt intervention by a doctor should this be deemed necessary.

In another aspect, the invention features an apparatus for monitoring the fluorescence emitted from the surface of the biological tissue. Such an apparatus includes an excitation source suitable for emitting excitation radiation in order to excite a fluorescence marker, a camera comprising a sensor of the fluorescence light emitted from the surface of the biological tissue, under the effect of the excitation radiation, a computer for recording and storing fluorescence images captured by the camera, and for processing the fluorescence images, and a screen for displaying images resulting from the processing of the fluorescence images by the computer, the computer being configured to process fluorescence images using software for implementing the methods mentioned above.

In some embodiments, the apparatus includes a light source that illuminates in a spectral band to which a marker is sensitive, thereby exciting fluorescence of the marker. However fluorescence light sensor is not sensitive to this excitation spectral band. Instead, it senses radiation in the wavelength range that corresponds to that in which the marker emits fluorescence.

As used herein, “fluorescence signal” refers to a relative value of the signal measured in a pixel using a camera. The signal represents intensity of the fluorescence emission at a point in the tissue that corresponds to the pixel.

In another aspect, the invention features a method that includes monitoring diffusion over time of a fluorescent marker that has been injected into a biological tissue at an injection-time. Such a method includes using an excitation light source, exciting the fluorescent marker and, during an interval that begins after the injection-time and ends at an end-time, using a camera to acquire fluorescence images of an area of the biological tissue, wherein each of the fluorescence images corresponds to a set of pixels, wherein a value of a signal that represents an intensity of fluorescence emission at a point in the tissue is associated with each pixel in the set of pixels. Using the camera to acquire the fluorescence images comprises executing first and second image-acquisition sequences, the first image-acquisition sequence starting at a first start-time after the injection-time and the second image-acquisition sequence starting at a second start-time after the injection-time. The method also includes comparing first and second images, the first image being a result of having processed images from the first image-acquisition sequence and the second image being a result of having processed images from the second image-acquisition sequence and displaying, on a screen, a result of the comparison, the result being an image representative of the area of the biological tissue.

None of the foregoing steps are carried out entirely in the human mind and all of the steps are carried out in a non-abstract manner.

The claimed subject matter results in a technical improvement in a processing system. The improvement arises in part because the processing system is able to carry out a procedure that it could not otherwise carry out. The instructions used for causing a processor to carry out these instructions exist on a manufacture that comprises tangible and non-transitory media. Alternatively, the instructions can be carried out using hardware, firmware, or a combination of both. In either case, the execution of instructions is a physical process that consumes energy and generates waste heat. The methods described herein are restricted solely to non-abstract implementations. No abstract implementations have been described. Accordingly, the claims only read on non-abstract implementations. Anyone who construed the claim as if it read on an abstract implementation would therefore be construing the claim incorrectly. As used herein, Applicant, acting as his own lexicographer, hereby defines the term “non-abstract” and its cognates to mean the converse of “abstract,” where “abstract” means what the Supreme Court and lower courts have construed it to mean as of the filing of this application.

### BRIEF DESCRIPTION OF THE DRAWINGS

Other features and advantages of the invention will become apparent on reading the following detailed description, and from the appended drawings. In these drawings:

FIG. 1 shows an apparatus for carrying out an imaging method.

FIG. 2 shows steps in an imaging method carried out by the apparatus of FIG. 1;

FIG. 3 shows the change in average intensity of a fluorescence signal over time in a region of tissue;

FIG. 4 shows process of a fluorescence signal in an area of tissue;

FIG. 5 shows intensity of a fluorescence signal in an area of tissue as it changes over time and having been normalized by a threshold that has been assumed to correspond to a healthy foot; and

FIG. 6 shows an overview of results like those in FIGS. 3-5 but with acquisition times separated by unequal time intervals.

### DETAILED DESCRIPTION

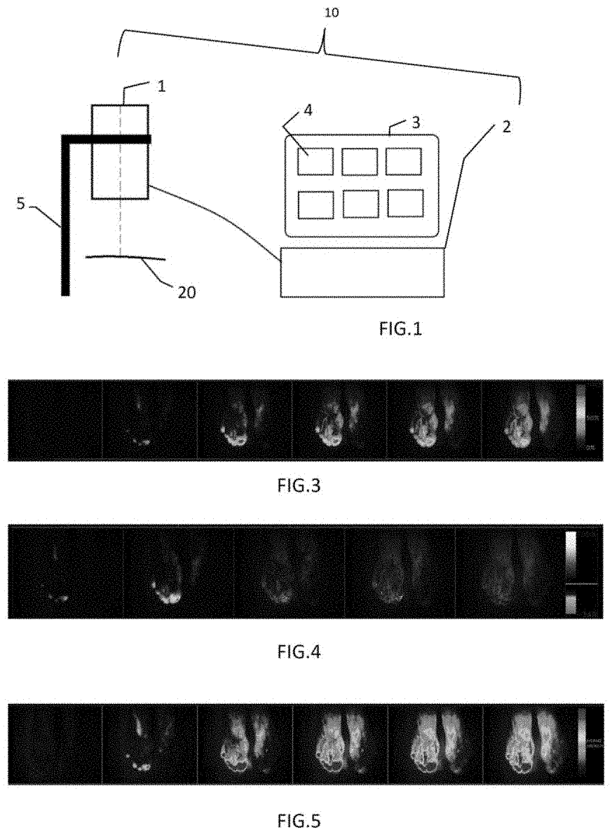

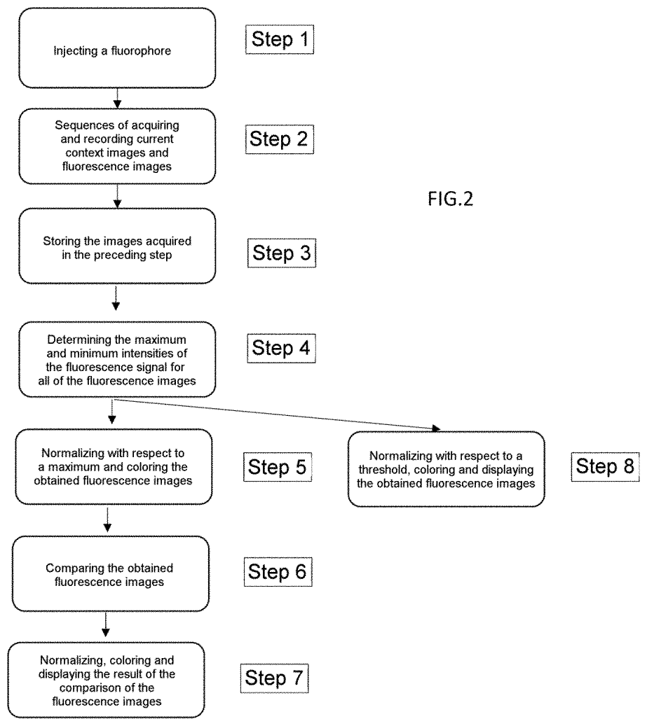

In a device 10 for monitoring the fluorescence emitted from the surface of the biological tissue 20, as shown in FIG. 1, a supporting arm 5 holds a probe 1 at a substantially constant distance from an area of biological tissue 20 that is to be observed. This distance may change periodically as a result of, for example, a patient's breathing.

In the embodiment described herein, the probe 1 includes a camera that captures fluorescence images. A useful camera is one that captures images in the near infrared. However, other cameras can be used depending on the wavelength of the fluorescence.

The probe 1 includes a sensor suitable for capturing images in the wavelengths emitted by fluorescent markers. As a result, the camera obtains an image that results from fluorescent light emitted by a fluorophore from the surface of an area of biological tissue 20.

The probe 1 also includes an excitation source suitable for emitting excitation radiation for exciting a fluorescence marker or fluorophore. A suitable source is a laser.

In some embodiments, the probe 1 comprises first and second cameras. The first camera is fluorescence camera that captures current images in the relevant wavelength band, for example, in the near infrared. The second camera captures current images in visible light.

As used herein, “fluorescence image” refers to an image of the fluorescence signal emitted from the surface of the biological tissue 20 that is being observed. The fluorescence image is captured using a fluorescence camera.

As used herein, a “current image” refers to an image extracted directly, without integration or summation with one or more other images, from a video produced using the probe's camera. Methods for producing a “current image” include illuminating the tissue 20 using light-emitting diodes that emit in the near infrared, or more generally, at whatever wavelengths the fluorescence camera is tuned to detect. Producing a current image can also be carried out by illuminating the tissue 20 using a light source suitable for exciting the fluorescent marker.

The device 10 also includes a computer 2 connected to the probe 1 and to a display 3 that displays images 4. The computer 2 records and stores the images captured by each camera and processes them. In some embodiments, it also processes the current images.

A process for using the device 10 begins with an intravenous injection of a fluorescent tracer, or fluorophore, at time T₀. Shortly thereafter, at time T₁, the probe 1, and in particular, the probe's fluorescence camera, begins to record an emission signal from the fluorophore is captured by the “fluorescence” camera of the probe 1 while the excitation source is left turned on. Recording continues until a final time T_{F} that has been selected to allow enough time for most of the progress of perfusion to be observed.

Software controls the acquisition of current and fluorescence image sequences. Some practices include presetting a time interval Δt between each sequence. Among these practices are those in which the time interval Δt remains constant. A suitable time interval is twenty seconds. Upon the lapse of the time interval, the software orders the acquisition of a sequence of one or more current images and of fluorescence images.

To promote measurement accuracy of the fluorescence signal corresponding to a sequence, it is useful to have the sequence be composed of fluorescence images acquired with different exposure times, different gains, or a combination of different exposure times and different gains. The time interval between the first and the last images of each of these sequences is chosen to be short enough so that the fluorescence signal may be considered to remain static throughout the progress made by the perfusion. In other words, the time taken to acquire each sequence is short enough to be considered negligible when compared to the rate of change of the measured signal.

A sequence includes some combination of the following first through fourth subsequences or series.

The first three subsequences are sequences of fluorescence images in which either exposure time or gain varies from one subsequence to the next. In general, these subsequences will have different numbers of images.

For example, a first subsequence could include N fluorescence images with an exposure time of X seconds, a second subsequences could include M fluorescence images with double the exposure time, i.e., 2X seconds, and a third subsequence could be a sequence of P fluorescence images with triple the exposure time, i.e. 3X seconds. The values N, M, and P are integers and can be all different, all the same, or some combination of both.

The fourth subsequences is a current image that is produced with the excitation source having been turned off and with lighting being provided by light-emitting diodes emitting in the infrared. This provides a way to obtain a current background image/c untainted by any fluorescence.

In some practices, the fourth subsequences is generated with the excitation source still turned on. In these embodiments, processing is carried out to find a current image. Such processing may include, for example, subtracting an image obtained with only the excitation source turned on from an image obtained with the excitation source and the lighting comprising light-emitting diodes both turned on.

In such practices, the excitation source always remains turned on and an image without the excitation source is obtained by subtracting an image that is not being lit by the light-emitting diodes from an image that is being lit by the diodes.

In some practices, an average over some number of images is used instead of an image.

Increasing exposure time, as carried out in the second and third subsequences, tends to reveal a signal that might otherwise remain embedded in noise when only the exposure time of the first subsequence is available. In the case of a linear camera, doubling the exposure time amounts to doubling the average signal level. Such a camera thus makes it easy to make exposure levels of all images correspond, even when using different exposure times. This type of sequence thus promotes an increase in precision of the fluorescent signal measurement and attenuation of noise as a result of an averaging effect on Poisson noise.

Preferred practices include summing grayscales obtained for each exposure time on a pixel-by-pixel basis. Before such summation, it is particularly useful to align the images with each other and to remove those pixels that correspond to saturated signal levels. Although a standard image-alignment process can be carried out, preferred alignment algorithms are those that have three degrees-of-freedom for rotation and another three degrees-of-freedom for translation and those that use singular points, optical flow or similar techniques.

The process includes counting the total exposure time per pixel and doing so either without taking into account exposure times corresponding to saturation of the pixel in question or by taking into account the exposure times of these pixels but replacing the signal value for these pixels with a value that has been extrapolated from values obtained from images in which the signal is not saturated. The sum of the grayscales per pixel is divided by the total exposure time corresponding to the pixel. This results in normalized images for a given exposure time.

Some practices also include applying weights or performing a non-linear color conversion using color tables. These practices are useful for managing signal variations having a large dynamic range. The result is that of slightly boosting signal strengths of weak signals while attenuating the strength of the strongest signals. This permits the signal to be displayed in a way that shows its entire range of amplitude variation while also avoiding either saturation or underexposure.

In the images thus obtained, accuracy, especially with respect to weak signals, is higher. Specifically, as indicated above, increasing the exposure time allows the signal-of-interest to emerge from the background noise. This is more particularly the case with a “fluorescence” camera fitted with a sensor that relies on a charge-coupled device. Thus, extending exposure time permits weak signals to emerge from the background noise. The computer, through appropriate processing, it possible to reduce noise by averaging Poisson noise in the image.

A more detailed example of a mode of implementation of the method according to the invention is described below.

The times Tᵢ at which the fluorescence information must be captured (with i ranging from 0 to F; F being the number of images on which the practitioner wishes to carry out his examination; in the example described here F=6), are entered as parameters in the software.

This acquisition then comprises, for example, the following steps:

Step 1: A stopwatch is started. At T₀, the practitioner injects ICG (indocyanine-green) and initiates image acquisition using the software.

Step 2: At a time T₁ after or equal to T₀, the software triggers the following operations:

Acquiring and recording a current image of the context or a background image IC₁; during this acquisition, the fluorescence excitation source remains turned off; depending on the type of probe, as mentioned above, the current background image IC₁ may for example be acquired using either a camera provided with a sensor of visible light, or using a camera equipped with a sensor of near infrared light (in the latter case, the laser excitation source is preferably turned off and lighting comprising light-emitting diodes emitting in the infrared is turned on);

Acquiring and recording a sequence of fluorescence images (the laser excitation source is turned on and the lighting comprising the light-emitting diodes emitting in the infrared is preferably turned off; if it is not, this substep is carried out as indicated above); as indicated above, this acquisition may possibly comprise acquiring series of images corresponding to different exposure times (e.g. a series of N images with an exposure time X, then one or more series ofM images corresponding to one or more exposure times Y greater than X (or different gains), in order to produce an HDR image (HDR being the acronym of high dynamic range) and achieve a better signal-to-noise ratio.

Computing a fluorescence image I₁ resulting from the sum of the fluorescence images processed as indicated above (alignment of the images with one another, removal or replacement of the saturated pixels, normalization for a given exposure time). Instead of summing the images, it is possible, in various variants, to aggregate the images, and to do this linear operations, weightings, etc., may or may not be used;

This image I₁ therefore represents a “freeze frame” of the perfusion level at the time T₁.

The operations of step 2 are repeated when each time Tᵢ (with this time i=2 to F) is reached, until ending with the acquisition at the time T_{F}.

Step 3: At the end, after the last acquisition at the time T_{F}, the various fluorescence levels corresponding to fluorescence images Iᵢ, and the current background images ICi, are stored in memory in the computer 2 (alternatively, images are stored in memory in the computer during the acquisition).

Step 4: The software then determines a maximum value of the intensity of the fluorescence signal for all of the fluorescence images Iᵢ. This maximum intensity value may be chosen by using an x^{th} percentile, x % of the maximum value and/or by smoothing the fluorescence images Iᵢ to avoid point artifacts on each of these fluorescence images L.

Step 5: The software then normalizes each fluorescence image Iᵢ with respect to the maximum determined in the previous step. Then, the software colors the fluorescence images Iᵢ thus normalized using a specific color conversion table. Then the software superimposes this result on the current context image corresponding to this time, leaving in grayscale any pixels of the current context image that do not exhibit fluorescence. An example of a series of fluorescence images Iᵢ obtained using the above method is shown in FIG. 3. Such a series of fluorescence images Iᵢ corresponds to an examination of the vascularization of a healthy foot.

Step 6: However, it is also possible to even further accentuate the progress of the fluorescence signals over time. To do this, the software makes it possible to compare with one another the intensity levels of temporally successive fluorescence images Iᵢ. Thus, the intensity of the fluorescence image Iᵢ+1 taken at Tᵢ+1 may be subtracted (after alignment), pixel-by-pixel, from the intensity of the fluorescence image L taken at Tᵢ. This difference makes it possible to highlight what happened between the time Tᵢ and the time Tᵢ+1.

It may be noted that simply subtracting the intensities of the fluorescence signal associated with each pixel is not the only way of highlighting the progress of a signal. Generally, the software can compute the difference between the square of the intensities, or even the difference between logarithms of the intensities, etc. or any distance, in the mathematical sense of the term, and more particularly an algebraic distance, between two successive fluorescence images. Thus, the computation on which the comparing operation is based comprises at least one operation chosen from the following operations: a subtraction between values of a signal representative of the intensity of the fluorescence emission, a computation of a norm of a quantity represented by values of a signal representative of the intensity of the fluorescence emission, a computation of an algebraic distance between values of a signal representative of the intensity of the fluorescence emission, and an “or” or “nor” or “xor” logic operation on values of a signal representative of the intensity of the fluorescence emission. Specifically, these logic operations may allow the following effects to be highlighted:
There is no fluorescence signal associated with a pixel or with a group of pixels, neither in the image resulting from the first acquisition sequence, nor in the image resulting from the second acquisition sequence (“NOR” operation): this reveals areas that are never vascularized.
There is a fluorescence signal associated with a pixel or with a group of pixels, either in the image resulting from the first acquisition sequence, or in the image resulting from the second acquisition sequence (“OR” operation): this reveals areas that are not vascularized at different times.
There is a fluorescence signal associated with a pixel or with a group of pixels, only in the image resulting from the first acquisition sequence, or in the image resulting from the second acquisition sequence (“XOR” operation): this reveals areas that are vascularized during only one of the sequences.

To be able to compare fluorescence images Iᵢ with one another, it is necessary for the fluorescence images Iᵢ to be aligned with one another so that the pixels in the various fluorescence images Iᵢ correspond. Thus, the difference between the images (or more generally the operation that allows the images to be compared with one another) may be computed pixel-by-pixel.

It will be noted that these comparing operations do not employ, in the computation, a reference image (i.e. a base line image) acquired for example before the appearance of fluorescence, i.e. an image that would in particular be subtracted from each image resulting from a sequence. Specifically, in particular during an operation in which two images resulting from two sequences are compared, the use of such a reference image is pointless since the corresponding information disappears during the subtraction employed in the comparing operation.

Step 7: Once this computation has been carried out by the software, for successive pairs of images, the software determines the maximum and minimum values thus obtained by computing the distance (in the sense indicated above) between the successive fluorescence images. These maximum and minimum values may possibly be positive or negative. Pixels given a negative value of the distance computed previously correspond to areas in which the fluorescence signal is weaker at the time Tᵢ+1 than at the time Tᵢ and conversely a positive value corresponds to an increase in the fluorescence in this location, during the corresponding time interval.

The software therefore normalizes the positive pixels of the images obtained via the preceding distance computation, with the maximum obtained for all the computations carried out on the fluorescence images of a sequence from T₁ to T_{F}. Likewise, the software normalizes the negative pixels of the images obtained via the preceding distance computation, with the minimum obtained for all the computations carried out on the images of a sequence from T₁ to T_{F}.

The software colors the images thus normalized with a specific false color for positive pixels (for example warm colors, from yellow to red) and a specific false color for negative pixels (for example cold colors from blue to purple). The software makes it possible to display the result obtained (see FIG. 4), by superimposing each colored image on the corresponding current context image. Such a presentation of the results makes it possible to give rapid visual information on the areas corresponding to an increasing signal, from when this signal increased in the areas in question (therefore with what delay), over which period of time, etc. Likewise, this type of presentation of the results makes it possible to give rapid visual information on the areas corresponding to a decreasing signal, from when it decreased, over what period of time, etc.

This type of display may in particular make it possible to visualize arterial or venous problems by identifying areas in which the intensity of the fluorescence signal decreases later than in others.

Step 8: In addition, the software makes it possible to normalize all of the fluorescence images L by a threshold defined and set in advance. The software then colors the images thus normalized using a color conversion table. For example, this conversion table is the same as that used in step 5 above.

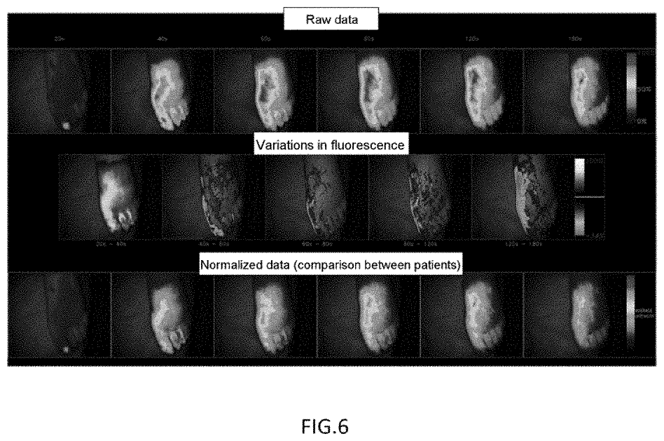

The software superimposes each image thus normalized on the corresponding current background image ICi. The result is displayed. This display makes it possible to compare perfusion progress between patients and to characterize it locally on the current image (obtained in the visible or near infrared for example). Specifically, for example, the threshold may be chosen so as to correspond to a standard average level for a healthy foot. A display in false colors then makes it possible to quickly visualize whether the average fluorescence intensity level measured for a patient is standard, or whether it is higher or lower (see FIG. 5).

The following convention may for example be chosen: if the colors are warm it means that the area is correctly perfused with respect to a standard level of perfusion. Conversely, cold colors indicate a lower than average vascularization.

All of the computed images may be displayed simultaneously (FIGS. 3, 4 and 5 are displayed together, for example in a manner analogous to FIG. 6).

As a variant, the time interval Δt between each sequence may not be regular. For example, this time interval Δt may be equal to twenty seconds, then forty seconds, then sixty seconds. An example of the result obtained with variable intervals Δt is shown in FIG. 6. In this case, advantageously, the normalization of the resulting image must take into account this variation in the time intervals.

The method according to the invention therefore makes it possible to facilitate the interpretation of the measurements of a fluorescence signal by way of a visual representation, that in particular provides precise information on the local progress of the fluorescence signals, and in particular on the following parameters:
delay of appearance of the fluorescence signal,
variation in the intensity of the fluorescence signal over time,
location of increases and decreases in the fluorescence signal over time.

The method according to the invention allows patients to be compared.

The method according to the invention provides an automatic analysis tool, in particular as it employs normalization with respect to a reference threshold (see the example of a healthy foot above) and not an arbitrary choice or intervention by a practitioner who could (in particular by poorly choosing the reference tissue) introduce bias, and therefore errors in the interpretation of the results. It also allows cases where there is a manifest error in the computation to be rapidly seen, which would not be possible with a curve (for example at the edges of a foot if the scene moved too much and/or if it is not/cannot any longer be aligned, for example if the foot leaves the field of view, or if an object passes through the field and causes a measurement artifact). However, the image alignment makes it possible to easily and effectively manage scenes that may move over time (in particular the movement of a patient's feet). The method according to the invention therefore makes it possible to work on tissues that are deformable and/or that may move over time. By virtue in particular of the detection of saturated pixels and of the normalization of the images, it allows over-exposures to be effectively managed. By virtue in particular of the combination of fluorescence images taken with different exposure times or gains, it allows under-exposures to be effectively managed. It allows a rapid comparison of the various areas of a tissue and between tissues of the same type.

1-7. (canceled)

8. A method comprising monitoring diffusion over time of a fluorescent marker that has been injected into a biological tissue at an injection-time, said method comprising using an excitation light source, exciting said fluorescent marker and, during an interval that begins after said injection-time and ends at an end-time, using a camera to acquire fluorescence images of an area of said biological tissue, wherein each of said fluorescence images corresponds to a set of pixels, wherein a value of a signal that represents an intensity of fluorescence emission at a point in said tissue is associated with each pixel in said set of pixels, wherein using said camera to acquire said fluorescence images comprises executing first and second image-acquisition sequences, said first image-acquisition sequence starting at a first start-time after said injection-time and said second image-acquisition sequence starting at a second start-time after said injection-time, wherein said method further comprises comparing first and second images, said first image being a result of having processed images from said first image-acquisition sequence and said second image being a result of having processed images from said second image-acquisition sequence and displaying, on a screen, a result of said comparison, said result being an image representative of said area of said biological tissue.

9. The method of claim 8, wherein comparing said first and second images comprises subtracting values of a signal representative of said intensity of said fluorescence emission.

10. The method of claim 8, wherein comparing said first and second images comprises computing a norm of a quantity represented by values of a signal representative of said intensity of said fluorescence emission.

11. The method of claim 8, wherein comparing said first and second images comprises computing an algebraic distance between values of a signal representative of said intensity of said fluorescence emission.

12. The method of claim 8, wherein comparing said first and second images comprises executing a logic operation on values of a signal representative of said intensity of said fluorescence emission, said logic operation being selected from the group consisting of “or,” “nor,” and “xor.”

13. The method of claim 8, wherein processing of said fluorescence images is carried out for a given series of fluorescence images in order to align them with one another before summing them pixel-by-pixel.

14. The method of claim 8, wherein executing an image acquisition sequence comprises acquiring a current background image, said method further comprising using said current background image to locate dynamic variations in said fluorescence signal in said area of said biological tissue when displaying said fluorescence images.

15. The method of claim 8, wherein executing an image-acquisition sequence comprises acquiring plural series of fluorescence images, each of said series having different exposure times.

16. The method of claim 8, wherein executing an image-acquisition sequence comprises acquiring plural series of fluorescence images, each of said series having different gains.

17. An apparatus for monitoring fluorescence emitted from a surface of a biological tissue, said apparatus comprising an excitation source, a camera, a computer, and a screen, wherein said excitation source is configured for emitting excitation radiation that excites a fluorescence marker, wherein said camera comprises a sensor that senses fluorescence light emitted as a result of said excitation radiation, wherein said computer is configured to record, store, images captured by said camera and to execute computer-readable instructions for processing said images by carrying out said method recited in claim 1, and wherein said screen is configured to display images that result from said computer having processed said fluorescence images.

18. The apparatus of claim 17, wherein said sensor is insensitive to said excitation radiation.
